(19) United States
(12) Patent Application Publication
(10) Pub. No.: US 2022/0389437 A1
(43) Pub. Date: Dec. 8, 2022

(54) METHODS OF IN PLANTA TRANSFORMATION USING AXILLARY MERISTEM

(71) Applicant: SYNGENTA CROP PROTECTION AG, Basel (CH)

(72) Inventors: Changbao Li, Research Triangle Park, NC (US); Wenjin Yu, Research Triangle Park, NC (US); Heng Zhong, Research Triangle Park, NC (US); Hua-ping Zhou, Research Triangle Park, NC (US)

(73) Assignee: SYNGENTA CROP PROTECTION AG, Basel (CH)

(21) Appl. No.: 17/775,955

(22) PCT Filed: Nov. 24, 2020
(86) PCT No.: PCT/US2020/061899

§ 371 Date: May 11, 2022

Related U.S. Application Data
(60) Provisional application No. 62/940,268, filed on Nov. 26, 2019.

(51) Int. Cl. C12N 15/82

(52) U.S. Cl. CPC C12N 15/8212; C12N 15/8213; C12N 15/8205

(57) ABSTRACT

Conventional gene transformation requires tissue culture, and some elite lines have very low transformation efficiency in tissue culture. The disclosure relates to methods of in planta transformation. In some aspects, an axillary meristem of a plant is wounded and contacted with a transformation agent. The wounded axillary meristem is then regenerated and treated with a selection step, resulting in transformed tissue that can produce transgenic seeds.

### RELATED APPLICATIONS

This application claims priority from provisional application 62/940,268 filed Nov. 26, 2019 and incorporated by reference in its entirety herein.

### FIELD OF THE INVENTION

The invention relates to compositions and methods for transformation of plants, such as dicots. In particular, the invention relates to in planta methods of transformation.

### BACKGROUND

Genome editing is recognized as a revolution in plant breeding. Although significant progress has been made in multiple plant systems, there are still technical hurdles which need to be overcome. Most genome editing approaches rely on tissue culture. Tissue culture is time consuming and labor intensive. Conventional gene transformation also requires tissue culture, and some elite lines have very low transformation efficiency using this method. A tissue culture-free and genotype-independent method would significantly reduce the labor and time invested in crop genome editing and transformation.

### SUMMARY

The disclosure relates to methods of transformation. As described herein, in planta methods of transformation were developed by wounding axillary meristems of plants and applying transformation-inducing agents, e.g., Agrobacterium, to the wounded axillary meristems. Apical dominance was broken to allow the axillary meristems to regenerate into transformed axillary meristems. Cells within the wounded axillary meristem were shown to be successfully transformed. The transformed axillary meristems were then grown into shoots under selection (in planta selection) and those shoots were shown to be transformed. Whole plants grown from the transformed shoots were also shown to be transformed. The methods described herein were shown to be effective in multiple different dicots and in different germplasms. Without wishing to be bound by theory, unlike other conventional methods of transformation, which rely on embryogenesis, the methods described herein are thought to utilize organogenesis to generate transformed plants and plant parts. The methods described herein are useful, e.g., to introduce heterologous nucleic acids or proteins into plant cells for genome editing and transgenic plant generation.

In some aspects, the disclosure provides a method, comprising: a) providing a plant comprising an axillary meristem and a shoot apical meristem, b) removing or wounding at least part of the axillary meristem to produce a wounded axillary meristem region, c) contacting the wounded axillary meristem region with a heterologous polynucleotide and/or heterologous protein under conditions where the heterologous polynucleotide and/or heterologous protein enters wounded axillary meristem region, d) removing the shoot apical meristem or suppressing the growth of the shoot apical meristem at the same time as step b) or step c) or after step c), and e) growing the plant to regenerate at least part of the wounded axillary meristem region to produce a regenerated axillary meristem or shoot.

In some embodiments, the axillary meristem is two axillary meristems, the wounded axillary meristem area is two wounded axillary meristem areas, and the regenerated axillary meristem is two regenerated axillary meristems. In some embodiments, the method comprises removing or suppressing the shoot apical meristem at the same time as step b). In some embodiments, the method comprises removing or suppressing the shoot apical meristem after step c). In some embodiments, the shoot apical meristem is removed or suppressed 2-7 days, optionally 3-4 days, after the contacting. In some embodiments, the plant is a dicot plant, optionally a soy plant, a tobacco plant, a bean plant, a sunflower plant, a cotton plant, a tomato plant, a watermelon plant, a squash plant, a cucumber plant, a lettuce plant or a pepper plant. In some embodiments, step c) comprises contacting the wounded axillary meristem region with a heterologous polynucleotide, wherein the heterologous polynucleotide comprises a selectable marker and wherein the method further comprises contacting the plant with a selection agent to eliminate or reduce untransformed tissue, wherein at least part of the contacting with the selection agent occurs during or after step e). In some embodiments, the contacting with the selection agent comprises (i) adding the selection agent to a medium in which the plant is growing, (ii) spraying the plant with the selection agent, or (iii) applying the selection agent to the wounded axillary meristem region and/or regenerated axillary meristem, or a combination thereof, optionally wherein the combination thereof is a combination of (i) and (iii). In some embodiments, the contacting with the selection agent occurs for a period of time, optionally for at least one week, further optionally between 3-5 weeks. In some embodiments, the selection agent is an herbicide, an antibiotic, or a non-metabolizable sugar. In some embodiments, the selection agent is glyphosate, glufosinate, spectinomycin, bensulfuron-methyl, imazapyr, D-xylose, mannose or kanamycin. In some embodiments, the method further comprises performing an assay on the regenerated axillary meristem or a sample of the regenerated axillary meristem to assess for the presence or absence of transformed cells and/or to assess for the number of transformed cells. In some embodiments, the method further comprises growing the plant to produce a seed and harvesting the seed, wherein the seed optionally comprises at least part of the heterologous polynucleotide. In some embodiments, the method further comprises growing the seed to produce a progeny plant, optionally wherein the progeny plant comprises at least part of the heterologous polynucleotide. In some embodiments, the heterologous polynucleotide encodes or comprises a genome editing agent or wherein the heterologous protein comprises a genome editing agent, optionally wherein the genome editing agent is a nuclease or a recombinase. In some embodiments, the heterologous polynucleotide comprises one or more polynucleotides encoding a Cas protein and/or a guide RNA or wherein the heterologous protein comprises a Cas protein, optionally wherein the Cas protein is Cas9 or Cas12a, or a functional variant thereof. In some embodiments, the heterologous polynucleotide comprises an expression cassette comprising a coding sequence. In some embodiments, the expression cassette further comprises a promoter operably linked to the coding sequence. In some embodiments, the coding sequence encodes a protein or non-coding RNA of interest. In some embodiments, wherein the contacting in step c) is performed with Agrobacterium, viral particles, microparticles, nanoparticles, cell membrane penetrating peptides, aerosol beam, chemicals, electroporation, or pressure. In some embodiments, the contacting is performed with Agrobacterium or viral particles and the contacting comprises an infection step and an incubation step. In some embodiments, the infection step is performed for 30 minutes to 24 hours, optionally 1-9 or 5-12 hours, and the incubation step is performed in darkness for at least 2 days, optionally 3-7 days. In some embodiments, the plant is between 1-30 days old, optionally 4-7 days old. In some embodiments, the axillary meristem is a cotyledonary axillary bud, or a meristem in an axil of a true leaf. In some embodiments, the method further comprises removing a cotyledon of the plant prior to removing or suppressing the shoot apical meristem. In some embodiments, the method further comprises growing the regenerated axillary meristem into a shoot.

In other aspects, the disclosure provides a plant or plant part produced by the method of any of the above-mentioned embodiments. In other aspects, the disclosure provides a plant or plant part produced by a method provided in the Examples. In other aspects, the disclosure provides a progeny seed produced by crossing the plant with a second plant or by selfing the plant. In other aspects, the disclosure provides a derivative or a commodity product produced or obtained from the plant or plant part.

### BRIEF DESCRIPTION OF THE DRAWINGS

FIG. 1 is a diagram showing an example in planta transformation process for soy

FIG. 2 shows CFP expression in axillary meristem cells after transformation with Agrobacterium.

FIG. 3 shows CFP expression in a regenerating axillary meristem after 7 days of selection with glyphosate.

FIG. 4 shows CFP expression in a regenerating axillary meristem after 14 days of selection with glyphosate.

FIG. 5 shows CFP expression in transgenic shoots.

FIG. 6 shows organogenesis of sunflower adventitious shoots from cotyoledonary areas.

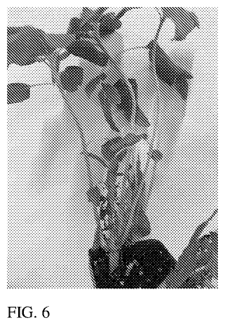

FIG. 7 shows the regenerated sunflower adventitious shoot produced a normal head.

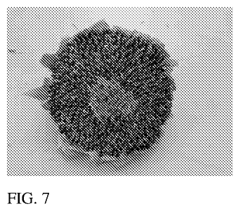

### DEFINITIONS

Although the following terms are believed to be well understood by one of ordinary skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

All patents, patent publications, non-patent publications referenced herein are incorporated by reference in their entireties for the teachings relevant to the sentence or paragraph in which the reference is presented. In case of a conflict in terminology, the present specification is controlling.

As used herein, the terms “a” or “an” or “the” may refer to one or more than one, unless the context clearly and unequivocally indicates otherwise. For example, “an” endogenous nucleic acid can mean one endogenous nucleic acid or a plurality of endogenous nucleic acids.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means ±1 ° C., preferably ±0.5° C. Where the term “about” is used in the context of this invention (e.g., in combinations with temperature or molecular weight values) the exact value (i.e., without “about”) is preferred.

As used herein, the term “apical dominance” refers to a phenomenon by which a main shoot dominates and inhibits the growth of axillary meristems. Apical dominance is thought to be caused by auxin, which moves downward toward the axillary meristems and inhibits their growth.

As used herein, the term “axillary bud” means an embryonic or organogenic bud located in the axil of a cotyledon or leaf. The axillary bud contains axillary meristem which is capable of developing into a branch shoot or flower clusters.

As used herein, the term “axillary meristem” refers to a region of a plant containing stem cells that is located on the lateral side of a stem of a plant, but is not located at the apex of the stem.

“Explant,” as used herein, refers to tissue, a piece of tissue, or pieces of tissue derived from a plant or a plant part, such as a seed. An explant can be a part of a plant, such as immature embryos, leaves meristems, or can be derived from a portion of the shoot, leaves, immature embryos or any other tissue of a plant or seed.

As used herein, the term “expression cassette” refers to a nucleotide capable of directing expression of a particular nucleic acid sequence in a host cell. In some embodiments, the expression cassette comprises, consists essentially of or consists of one or more promoter sequences (e.g., one or more constitutive/inducible promoter sequences, one or more tissue- and/or organ-specific promoter sequences and/or one or more developmental stage-specific promoter sequences) operably linked to a nucleic acid of interest, which is operably linked to a termination sequence. Expression cassettes often comprise sequences required for proper translation of the nucleic acid sequence of interest in the host cell. The expression cassette may be chimeric in that at least one of its components is heterologous with respect to at least one of its other components. The expression cassette may be one that is naturally occurring but that has been obtained in a recombinant form useful for heterologous expression. Typically, however, the expression cassette is heterologous with respect to the host (i.e., the particular nucleic acid sequence of the expression cassette does not occur naturally in the host cell and must have been introduced into the host cell or an ancestor of the host cell by a transformation event).

As used herein, the term “genome editing agent” refers to an agent that is capable of inducing a deletion, insertion, indel, or other modification in the genome of a cell, e.g., by creating a single or double-stranded break in the genome. Examples of genome editing agents include CRISPR/Cas agents (e.g., Cas proteins and guide RNAs), transcription activator-like effector nucleases (TALENs), DNA-guided nucleases, meganucleases, recombinases, and zinc finger nucleases. Cas proteins include Cas9, Cas12a (also known as Cpf1), C2c1, C2c2, and C2c3, and functional variants thereof. Example Cas9 and Cas12a proteins include Streptococcus pyogenes Cas9 (SpCas9), Streptococcus thermophilus Cas9 (StCas9), Streptococcus pasteurianus (SpaCas9), Campylobacter jejuni Cas9 (CjCas9), Staphylococcus aureus (SaCas9), Francisella novicida Cas9 (FnCas9), Neisseria cinerea Cas9 (NcCas9), Neisseria meningitis Cas9 (NmCas9), Francisella novicida Cpf1 (FnCpf1), Acidaminococcus sp. Cpf1 (AsCpf1), or Lachnospiraceae bacterium ND2006 Cpf1 (LbCpf1). A “variant” of a Cas protein refers to a protein or polypeptide derivative of a wild type Cas protein, e.g., a protein having one or more point mutations, insertions, deletions, truncations, a fusion protein, or a combination thereof. In certain embodiments, the Cas variant is a functional variant which substantially retains the nuclease activity of or has better nuclease activity than the wild type Cas protein. Example guide RNAs include single guide RNAs and dual guide RNAs.

As used herein, the term “heterologous” refers to a polynucleotide/polypeptide at least a part of which originates from a foreign species, or, if from the same species, is substantially modified from its native form in composition and/or genomic locus by deliberate human intervention. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., present in a different copy number, and/or under the control of different regulatory sequences than that found in the native state of the nucleic acid molecule. A nucleic acid sequence can also be heterologous to other nucleic acid sequences with which it may be associated, for example in a nucleic acid construct, such as e.g., an expression vector. As one nonlimiting example, a promoter may be present in a nucleic acid construct in combination with one or more regulatory element and/or coding sequences that do not naturally occur in association with that particular promoter, i.e., they are heterologous to the promoter.

As used herein, the term “in planta” when referring to a process or method step refers to a process or method step that is performed on a plant and not on excised or in vitro cultivated plant tissues or organs. For clarity, a plant includes those that have been wounded or have had one or more tissues removed, e.g., a plant having wounded axillary meristems and/or removed SAMs.

As used herein, the term “in planta transformation” refers to a transformation method that is performed on a plant without any tissue culture steps performed on any excised tissues or organs. For clarity, tissue culture steps do not include growing the plant on or in growth media, hydroponics, media plates, etc.

The terms “nucleic acid” or “polynucleotide” are used interchangeably herein and refer to any physical string of monomer units that can be corresponded to a string of nucleotides, including a polymer of nucleotides (e.g., a typical DNA polymer or polydeoxyribonucleotide or RNA polymer or polyribonucleotide), modified oligonucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated. The nucleic acid can be present in a vector, such as in a cell, virus or plasmid.

As used herein, the phrases “operably linked,” “operatively linked,” “operatively associated” or “in operative association” and the like, mean that elements of a nucleic acid construct such as an expression cassette or nucleic acid molecule are configured so as to perform their usual function. Thus, regulatory or control sequences (e.g., promoters) operatively associated with a nucleotide sequence are capable of effecting expression of the nucleotide sequence. For example, a promoter is operably linked with a coding sequence or functional RNA when it is capable of affecting the expression of that coding sequence or functional RNA (i.e., the coding sequence or functional RNA is under the transcriptional control of the promoter). Coding sequences in sense or antisense orientation can be operably-linked to regulatory sequences. The control sequences need not be contiguous with the nucleotide sequence of interest, as long as they function to direct the expression thereof. Thus, for example, intervening untranslated, yet transcribed, sequences can be present between a promoter and a coding sequence, and the promoter sequence can still be considered “operably linked” to the coding sequence.

The term “plant” refers to any plant, particularly to agronomically useful plants (e.g. seed plants), and “plant cell” is a structural and physiological unit of the plant, which comprises a cell wall but may also refer to a protoplast. The plant cell may be in form of an isolated single cell or a cultured cell, or as a part of higher organized units such as for example, a plant tissue, or a plant organ differentiated into a structure that is present at any stage of a plant's development. A plant may be a monocotyledonous or dicotyledonous plant species. The term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as pollen, ovules, leaves, embryos, roots, root tips, anthers, flowers, fruits, stems, shoots, scions, rootstocks, seeds, protoplasts, calli, and the like. The term “plant part” also includes explants.

The term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

“Promoter” refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. The meaning of the term “promoter” includes “promoter regulatory sequences.”

As used herein, the term “shoot apical meristem”, “shoot apex meristem” or “SAM” refers to a region of a plant containing stem cells that is located at the apex of a stem of a plant.

By “stably introducing” or “stably introduced” in the context of a polynucleotide introduced into a cell is intended the introduced polynucleotide is stably incorporated into the genome of the cell, and thus the cell is stably transformed with the polynucleotide.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear, mitochondrial and the plastid genome, and therefore includes integration of the nucleic acid into, for example, the chloroplast genome. Stable transformation as used herein can also refer to a transgene that is maintained extrachromasomally, for example, as a minichromosome.

“Selection agent” refers to an agent (e.g., a chemical) that interacts with a selectable marker to give a plant cell a selective advantage. Example selection agents are known in the art and described herein, such as glyphosate, glufosinate, spectinomycin, bensulfuron-methyl, and kanamycin.

A “selectable marker” or “selectable marker gene” refers to a gene whose expression in a plant cell gives the cell a selective advantage. “Positive selection” refers to a transformed cell acquiring the ability to metabolize a substrate that it previously could not use or could not use efficiently, typically by being transformed with and expressing a positive selectable marker gene. This transformed cell thereby grows out of the mass of nontransformed tissue. Positive selection can be of many types from inactive forms of plant growth regulators that are then converted to active forms by the transferred enzyme to alternative carbohydrate sources that are not utilized efficiently by the nontransformed cells, for example mannose, which then become available upon transformation with an enzyme, for example phosphomannose isomerase, that allows them to be metabolized. Nontransformed cells either grow slowly in comparison to transformed cells or not at all. Other types of selection may be due to the cells transformed with the selectable marker gene gaining the ability to grow in presence of a negative selection agent, such as an antibiotic or an herbicide, compared to the ability to grow of non-transformed cells. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell (typically a transgene).

The term “transformation” as used herein refers to the transfer of a nucleic acid into a host cell, which includes integration into a chromosome, heritable extrachromosomal events and transient transfer. In some particular embodiments, the introduction into a plant, plant part and/or plant cell is via bacterial-mediated transformation, particle bombardment transformation (also called biolistic particle transformation), calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of a nucleic acid into the plant, plant part and/or cell thereof, or a combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (2002, Cell Mol Biol Lett 7:849-858 (2002)).

As used herein, the term “transgenic” refers to any plant, plant cell, callus, plant tissue, or plant part that contains all or part of at least one heterologous polynucleotide. In some embodiments, all or part of the heterologous polynucleotide is stably integrated into a chromosome or stable extra-chromosomal element, so that it is passed on to successive generations.

### DETAILED DESCRIPTION

Provided herein are methods and compositions for transforming a plant in planta and optionally performing one or more selection steps in planta.

In some aspects the disclosure provides a method, comprising (a) providing a plant comprising an axillary meristem (e.g., a cotyledonary axillary bud or a true leaf axillary meristem) and a shoot apical meristem, (b) removing or wounding (e.g., by cutting, piercing, or crushing) at least part of the axillary meristem to produce a wounded axillary meristem region, (c) contacting the wounded axillary meristem region with a heterologous polynucleotide and/or heterologous protein under conditions where the heterologous polynucleotide and/or heterologous protein enters the wounded axillary meristem region, (d) removing or suppressing the growth of the shoot apical meristem at the same time as step (b) or step (c) or after step (c), and (e) growing the plant to regenerate at least part of the wounded axillary meristem region to produce a regenerated axillary meristem.

In some embodiments of the method, the plant is a dicot plant. In some embodiments, the plant is a monocot plant. In some embodiments, the plant is a soy plant, a bean plant, a sunflower plant, a pepper plant, or a tobacco plant. In some embodiments, the plant is a soy plant.

In some embodiments of the method, the axillary meristem is one, two, three, four, or more axillary meristems and at least one of the axillary meristems in wounded. In some embodiments, all of the axillary meristems are wounded. In some embodiments, e.g., in a dicot plant, the axillary meristem is two axillary meristems and one or both of the axillary meristems are wounded.

In some embodiments of the method, the whole shoot apical meristem is removed. In some embodiments, the entire region above the epicotyl that includes the shoot apical meristem is removed. In some embodiments, a part of the apical meristem is removed (including by damaging the apical meristem), wherein the part removed is sufficient to break apical dominance In some embodiments, the method comprises removing or suppressing the growth of the shoot apical meristem after step (c). In some embodiments, the shoot apical meristem (in whole or in part) is removed 1, 2, 3, 4, 5, 6, 7, 8, 9, 10, 11, 12, 13, 14 or more days, e.g., 2-7 days, 2-6 days, 2-5 days, 2-4 days, 2-3 days, 3-7 days, 3-6 days, 3-5 days or 3-4 days, after step (c). In some embodiments, the method comprises removing or suppressing the growth of the apical meristem at the same time as (e.g., during at least some part of) step (b) or step (c). In some embodiments, suppressing the growth of the apical meristem comprises killing cells of the apical meristem or applying an inhibitor such that apical dominance is broken.

In some embodiments, plants, plant parts and plant cells transformed with a heterologous polynucleotide can be selected, e.g., using selectable markers present in the heterologous polynucleotide. In some embodiments, the plants, plant parts and plant cells transformed with a heterologous polynucleotide are selected using one or more selection steps or selection agents described in the Examples.

Examples of selectable markers include, but are not limited to, genes that provide resistance or tolerance to antibiotics such as kanamycin (Dekeyser et al. 1989, Plant Phys 90: 217-23), spectinomycin (Svab and Maliga 1993, Plant Mol Biol 14: 197-205), streptomycin (Maliga et al. 1988, Mol Gen Genet 214: 456-459), hygromycin B (Waldron et al. 1985, Plant Mol Biol 5: 103-108), bleomycin (Hille et al. 1986, Plant Mol Biol 7: 171-176), sulphonamides (Guerineau et al. 1990, Plant Mol Biol 15: 127-136), streptothricin (Jelenska et al. 2000, Plant Cell Rep 19: 298-303) , or chloramphenicol (De Block et al. 1984, EMBO J 3: 1681-1689). Other selectable markers include genes that provide resistance or tolerance to herbicides, such as the S4 and/or Hra mutations of acetolactate synthase (ALS) that confer resistance to herbicides including sulfonylureas, imidazolinones, triazolopyrimidines, and pyrimidinyl thiobenzoates; 5-enol-pyrovyl-shikimate-3-phosphate-synthase (EPSPS) genes, including but not limited to those described in U.S. Pat. Nos. 4,940,935, 5,188,642, 5,633,435, 6,566,587, 7,674,598 (as well as all related applications) and the glyphosate N-acetyltransferase (GAT) which confers resistance to glyphosate (Castle et al. 2004, Science 304:1151-1154, and U.S. Patent Application Publication Nos. 20070004912, 20050246798, and 20050060767); BAR which confers resistance to glufosinate (see e.g., U.S. Pat. No. 5,561,236); aryloxy alkanoate dioxygenase or AAD-1, AAD-12, or AAD-13 which confer resistance to 2,4-D; genes such as Pseudomonas HPPD which confer HPPD resistance; Sprotophorphyrinogen oxidase (PPO) mutants and variants, which confer resistance to peroxidizing herbicides including fomesafen, acifluorfen-sodium, oxyfluorfen, lactofen, fluthiacet-methyl, saflufenacil, flumioxazin, flumiclorac-pentyl, carfentrazone-ethyl, sulfentrazone,); and genes conferring resistance to dicamba, such as dicamba monoxygenase (Herman et al. 2005, J Biol Chem 280: 24759-24767 and U.S. Pat. No. 7,812,224 and related applications and patents). Other examples of selectable markers can be found in Sundar and Sakthivel (2008, J Plant Physiology 165: 1698-1716), herein incorporated by reference. Additional selectable markers for use in the disclosure are known in the art such as Phosphinothricin N-acetyl transferase (PAT) and Aminoglycoside 3′-adenylyiltransferase (aadA) (see, e.g., Rosellini (2012) Selectable Markers and Reporter Genes: A Well Furnished Toolbox for Plant Science and Genetic Engineering, Critical Reviews in Plant Sciences, 31:5, 401-453).

Other selection systems include using drugs, metabolite analogs, metabolic intermediates, and enzymes for positive selection or conditional positive selection of transgenic plants. Examples include, but are not limited to, a gene encoding phosphomannose isomerase (PMI) where mannose is the selection agent, or a gene encoding xylose isomerase where D-xylose is the selection agent (Haldrup et al. 1998, Plant Mol Biol 37: 287-96). Finally, other selection systems may use hormone-free medium as the selection agent. One non-limiting example the maize homeobox gene knl, whose ectopic expression results in a 3-fold increase in transformation efficiency (Luo et al. 2006, Plant Cell Rep 25: 403-409). Examples of various selectable markers and genes encoding them are disclosed in Miki and McHugh (J Biotechnol, 2004, 107: 193-232; incorporated by reference).

In some embodiments of the disclosure, the selectable marker may be plant derived. An example of a selectable marker which can be plant derived includes, but is not limited to, 5-enolpyruvylshikimate-3-phosphate synthase (EPSPS). The enzyme 5-enolpyruvylshikimate-3-phosphate synthase (EPSPS) catalyzes an essential step in the shikimate pathway common to aromatic amino acid biosynthesis in plants. The herbicide glyphosate inhibits EPSPS, thereby killing the plant. Transgenic glyphosate-tolerant plants can be created by the introduction of a modified EPSPS transgene which is not affected by glyphosate (for example, U.S. Pat. No. 6,040,497; incorporated by reference). Other examples of a modified plant EPSPS which can be used as a selectable marker in the presence of glyphosate includes a P106L mutant of rice EPSPS (Zhou et al 2006, Plant Physiol 140: 184-195) and a P106S mutation in goosegrass EPSPS (Baerson et al 2002, Plant Physiol 129: 1265-1275). Other sources of EPSPS which are not plant derived and can be used to confer glyphosate tolerance include but are not limited to an EPSPS P101S mutant from Salmonella typhimurium (Comai et al 1985, Nature 317: 741-744) and a mutated version of CP4 EPSPS from Agrobacterium sp. Strain CP4 (Funke et al 2006, PNAS 103: 13010-13015). Although the plant EPSPS gene is nuclear, the mature enzyme is localized in the chloroplast (Mousdale and Coggins 1985, Planta 163:241-249). EPSPS is synthesized as a preprotein containing a transit peptide, and the precursor is then transported into the chloroplast stroma and proteolytically processed to yield the mature enzyme (della-Cioppa et al. 1986, PNAS 83: 6873-6877). Therefore, to create a transgenic plant which has tolerance to glyphosate, a suitably mutated version of EPSPS which correctly translocates to the chloroplast could be introduced. Such a transgenic plant then has a native, genomic EPSPS gene as well as the mutated EPSPS transgene. Glyphosate could then be used as a selection agent during the transformation and regeneration process, whereby only those plants or plant tissue that are successfully transformed with the mutated EPSPS transgene survive.

In some embodiments of the method, the heterologous polynucleotide comprises a selectable marker and the method further comprises contacting the plant with a selection agent to eliminate or reduce untransformed tissue, wherein at least part of the contacting with the selection agent occurs during step (e). In some embodiments, the selection agent is an herbicide, an antibiotic, or a non-metabolizable sugar. In some embodiments, the selection agent is glyphosate, glufosinate, spectinomycin, bensulfuron-methyl, imazapyr, D-xylose, mannose or kanamycin. In some embodiments, the selectable marker is EPSPS and the selection agent is glyphosate.

In some embodiments of the method, the contacting with the selection agent comprises adding the selection agent to a medium (e.g., soil or hydroponics) in which the plant is growing (e.g., by watering or applying to the soil or other medium a composition comprising the selection agent, such as between 1 uM to 1M of a selection agent, e.g., 10 uM to 500 uM of glyphosate), spraying the plant with the selection agent (e.g., with a sprayable composition comprising the selection agent, such as 1 uM to 1M of a selection agent, e.g., between 10 uM to 50 mM glyphosate), or applying the selection agent (such as between 1 uM to 1M of a selection agent, e.g., 10 uM to 200 uM glyphosate or 1 uM to 10 uM Bensulfuron-methyl) to the regenerated axillary meristem (e.g., using a solution, gel, absorbable material (e.g., cotton ball) or other material that can release the selection agent (such as onto the wounded axillary meristem and/or regenerated axillary meristem). In some embodiments, the contacting with the selection agent occurs for at least one day, at least one week, at least two weeks, at least three weeks, at least four weeks, at least five weeks, or longer. In some embodiments, the contacting with the selection agent occurs for between 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, or 5-6 weeks. In some embodiments, the contacting with the selection agent occurs for between 1 day to 6 weeks. In some embodiments, the contacting with the selection agent occurs for between 3-6 weeks.

In some embodiments of the method, the method further comprises performing an assay on a sample of the regenerated axillary meristem to assess the presence or absence of transformed cells in the sample and/or to assess the number of transformed cells in the sample. Example assays include fluorescent protein detection, qPCR, real-time PCR, immunoassays, and the like.

In some embodiments of the method, the method further comprises growing the plant to produce a seed (e.g., one seed, two seeds, ten seeds, twenty seeds, fifty seeds or more) optionally comprising at least part of the heterologous polynucleotide and harvesting the seed. In some embodiments, all seeds produced by the plant comprise at least part of the heterologous polynucleotide. In some embodiments, at least one seed, or more seeds (e.g., at least 10%, at least 20%, at least 30%, at least 40%, at least 50%, at least 60%, at least 70%, at least 80%, or at least 90%) of the seeds, produced by the plant comprise at least part of the heterologous polynucleotide. In some embodiments of the method, the method further comprises growing the seed(s) to produce a progeny plant(s), optionally comprising at least part of the heterologous polynucleotide.

In some embodiments of the method, the heterologous polynucleotide encodes a genome editing agent, e.g., a CRISPR/Cas agent, a TALEN, a DNA-guided nuclease, a meganuclease, a recombinase, or a zinc finger nuclease. In some embodiments of the method, the heterologous protein comprises a genome editing agent, e.g., a Cas protein, a TALEN, a DNA-guided nuclease, a meganuclease, a recombinase, or a zinc finger nuclease. In some embodiments, the heterologous polynucleotide comprises one or more polynucleotides encoding a Cas protein and/or a guide RNA. In some embodiments, the heterologous polynucleotide comprises one or more guide RNAs, optionally wherein the heterologous polynucleotide is comprised within a ribonucleoprotein (RNP) with a Cas protein. In some embodiments, the Cas protein is Cas9 or Cas12a, or a functional variant thereof.

In some embodiments of the method, the heterologous polynucleotide comprises an expression cassette comprising a coding sequence. In some embodiments of the method, the coding sequence encodes a protein or non-coding RNA of interest. In some embodiments, the protein or non-coding RNA of interest confers one or more desired traits on a plant, such as enhanced growth, enhanced yield, drought tolerance, salt tolerance, herbicide tolerance, insect resistance, pest resistance, disease resistance, temperature tolerance, enhanced nitrogen utilization and the like. In some embodiments, the coding sequence encodes a genome editing agent, such as a Cas protein and/or a guide RNA. In some embodiments, the heterologous polynucleotide comprises a coding sequence encoding a protein or non-coding RNA of interest and a coding sequence a selection marker. In some embodiments of the method, the expression cassette further comprises a promoter operably linked to the coding sequence(s). The promoter may be, e.g., a constitutive promoter, a tissue-specific promoter, or an inducible promoter.

In some embodiments of the method, the contacting in step (c) is performed with Agrobacterium, viral particles, particles such as microparticles or nanoparticles (e.g., gold or tungsten microparticles or nanoparticles), cell membrane penetrating peptides, aerosol beam, chemicals, electroporation, or pressure (e.g., vacuum). In some embodiments, the contacting in step (c) is performed with Agrobacterium. In some embodiments, the contacting in step (c) is performed with viral particles. In some embodiments, the contacting in step (c) is performed with gold or tungsten particles, such as microparticles or nanoparticles. In some embodiments, the contacting in step (c) is performed with cell membrane penetrating peptides. In some embodiments, the contacting in step (c) is performed with an aerosol beam. In some embodiments, the contacting in step (c) is performed with chemicals. In some embodiments, the contacting in step (c) is performed with electroporation. In some embodiments, the contacting in step (c) is performed with pressure (e.g., vacuum).

In some embodiments of the method, the contacting is performed with Agrobacterium or viral particles and the contacting comprises an infection step and an incubation step. In some embodiments of the method, the infection step is performed for at least 30 minutes, e.g., 30 minutes to 24 hours, such as 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 hours, and the incubation step is performed in darkness or in light or in a light/dark cycle for at least 1 day, at least 2 days, at least 3 days, at least 4 days, at least 5 days, at least 6 days, at least 7 days or more, e.g., 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 days. In some embodiments, the infection step comprises contacting the wounded axillary meristem(s) with a solution, gel, absorbable material or other material that contains the Agrobacterium or viral particles. In some embodiments, the infection step occurs for 5-12 hours. In some embodiments, the incubation step is performed in darkness for 3-7 days. In some embodiments, after incubation, antibiotics (e.g., Timentin, Cefotaxime and/or Vancomycin) are applied to eliminate the Agrobacterium or viral particles.

Agrobacterium-mediated transformation is a commonly used method for transforming plants because of its relatively high efficiency and increased throughput of transformation and because of its broad utility with many different species. Agrobacterium-mediated transformation typically involves transfer of a binary vector carrying the foreign DNA of interest to an appropriate Agrobacterium strain that may depend on the complement of vir genes carried by the host Agrobacterium strain either on a co-resident Ti plasmid or chromosomally (see, e.g., Uknes et al 1993, Plant Cell 5:159-169). The transfer of the recombinant binary vector to Agrobacterium can be accomplished, e.g., by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (see, e.g., Hofgen and Willmitzer 1988, Nucleic Acids Res 16:9877). Transformation of a plant by recombinant Agrobacterium usually involves incubation of the Agrobacterium with explants from the plant, although in the present disclosure the incubation occurs on the wounded axillary meristem(s). Transformed tissue is typically regenerated in the presence of a selection agent for a selectable marker that is located between the binary plasmid T-DNA borders.

In some embodiments of the method, the plant is between 1-100 days old, such as 1-30 days old, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days old, such as 4-7 days old. In some embodiments of the method, the method further comprises removing a cotyledon (e.g., one or both cotyledons) of the plant prior to removing or suppressing the growth of the shoot apical meristem. In some embodiments, the removing of the cotyledon occurs at the same time as the wounding of the axillary meristem(s). In some embodiments, the shoot apical meristem is removed at least 1 day, e.g., at least 1 day, at least 2 days, at least 3 days, at least 4 days, at least 5 days, at least 6 days, or at least 7 days after the cotyledon is removed, such as between 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 days after the cotyledon is removed. In some embodiments, the shoot apical meristem is removed 3-7 days after the cotyledon is removed.

In some embodiments of the method, the method comprises removing or suppressing the growth of the shoot apical meristem at the same time as (e.g., during) step (b) and optionally applying a selective reagent (such as 1 uM to 1M of a selection agent, e.g., 10-200 uM glyphosate and/or 1 uM-10 uM Bensulfuron-methyl) and a plant hormone (such as 1-10 mg/L 6-Benzylaminopurine) to the plant to suppress non-transformed cell division and stimulate transformed cell regeneration.

Other aspects of the disclosure relate to a plant or plant part produced by any of the methods described above or elsewhere herein, including in the Examples. Other aspects of the disclosure relate to progeny seed produced by crossing the plant produced by any of the methods described above or elsewhere herein with a second plant or by selfing the plant. Other aspects of the disclosure relate to a derivative or a commodity product produced or obtained from the plant or plant part produced by any of the methods described above or elsewhere herein. In some embodiments, the commodity product is selected from the group consisting of whole or processed seeds, flour, protein isolates, concentrates, liquids, syrups, pastes, sauces or other food or product produced from the plant or plant part.

Hereinafter, the present invention will be described in detail by the following examples. However, the following examples are illustrative of the present invention, and the scope of the present invention is not limited by the following examples.

### EXAMPLES

### Example 1: An Example Process for in Planta Transformation

Dicot embryos include an epicotyl (shoot apical meristem), a radicle, a hypocotyl and two cotyledons. However, this is another very tiny structure located at the axil between each cotyledon and the shoot. These structures are axillary meristems and are referred to as cotyledon axillary meristems (or cotyledon axillary buds at seedling stage). Such cotyledon buds have strong meristematic abilities, especially when the main shoot is removed, thus providing excellent materials for in planta transformation, which can be used for genome editing and transgenic plant generation.

A first example process for in planta is described below.
1) After seed germination or plant in seedling stage, remove or damage axillary meristems located in the axil of cotyledons or true leaves to create severe wounds. To break the dormancy of the axillary meristem, the primary shoot meristem is removed at the same time, or several days' later.
2) A transformation agent is used to facilitate transfer of the construct into the wounded meristem(s). An example is to apply Agrobacterium or virus containing construct onto the surface of the wounded area. Another example is to use biolistics or cell membrane penetrating peptides to introduce the construct.
3) After 1-7 days incubation under darkness or weak light, some cells within wounded meristem tissues will be transformed. Apply negative or positive selection agents to suppress non-transformed cell division and facilitate transformed cell proliferation. The selection agents are applied on the wounded areas (“top selection”), and/or watered into plant growth media or soil (“bottom selection”).
4) Keep plants under selection conditions for several weeks. The transformed cells proliferate and develop into transgenic shoots, while the non-transgenic shoots are suppressed by selection and severely stunted. The transformants can be verified with molecular- or bio-assays.
5) Grow the positives for (T1) seed production and identify germline transformation from produced seeds.

### Example 2: In Planta Transformation of Tobacco (Nicotiana benthamiana) Seedlings with Construct Containing AmCyan and EPSPS

### Methods

1) Tobacco seeds were sown in 2.5 inch soil pots. After germination, any additional plants were removed such that there was only seedling per pot. About 3-weeks-old seedlings grown from the seeds were used for axillary meristem transformation.
2) Wound axillary meristems: axillary meristems were wounded or partially removed using a blade or tweezers. The primary shoot apex meristem was then completely removed immediately thereafter to eliminate apex dominancy
3) Agrobacterium infection: Agrobacterium solution (OD=1) was prepared at the pH 5-6 for infection. The Agrobacterium harbored the construct containing the AmCyan and EPSPS genes. 100 uM acetosyringone was added into Agrobacterium solution before the incubation of bacteria on plants to increase infection efficiency. For the infection, tiny cotton balls were soaked with Agrobacterium solution and mounted on the wounded areas for 7 hours. After infection, excess Agrobacterium solution was removed using a piece of filter paper or paper towel.
4) Incubation: Flat trays containing the infected seedlings were covered with a dome to keep in moisture. The seedlings were placed under darkness for 3-7 days for incubation with the Agrobacterium. After incubation, antibiotics were applied (Timentin, Cefotaxime and Vancomycin) to inhibit the growth of Agrobacterium.
5) Selection: After incubation, the seedlings were moved into a growth chamber under 16 hours light and 8 hours dark conditions. Then a tiny cotton ball,soaked with selection solution containing 50-100 uM glyphos ate, was mounted on the infected axil area. The trays were covered with a dome to maintain high humidity. For some plants, at the same time or 1 week later, 300-500 uM glyphosate selection was watered into soil pots.
6) Cotton ball based “top selection” lasted for 2-4 weeks. The cotton balls were changed 1-2 times weekly. The “bottom selection” soil watering was done once a week, for 4-7 weeks. After 3-7 weeks of selection, non-transgenic cells were suppressed, and the transgenic cells were proliferated and developed into shoots.
7) Molecular analysis to identify the transgenic events: Leaf tissues were collected from putative transgenic shoots, and then DNA was extracted and analyzed for the presence of transgenes. Positive events were transplanted into new soil pots, grown under 16 hours day-length condition, and T1 seeds were harvested.
8) T1 seeds were sown and the transgene presence was detected in T1 generation based on Amcyan signals and molecular approach. The molecular approach was real time PCR, which was used to detect both Amcyan and EPSPS genes in putative transgenic plant tissue.

### Results

The results of the above method are shown in Tables 1 and 2 below. Top-selection-only methods using 50 or 100 uM glyphosate for 3 weeks did not produce positive events in tobacco, however it is expected that top selection should be feasible in tobacco by increasing the concentration and/or amount of time of the selection step. Bottom-selection-only method using 500 uM glyphosate selection for 6 weeks did not produce positive events in tobacco.

For Table 2, real time PCR was used to estimate transgenic copy numbers. In the 7 events tested, all were transgenic. The results showed that transgene segregation in T1 plants did not meet typical Mendel inheritance ratios. Without wishing to be bound by theory, the results suggested that some TO transgenic shoots could be of multicellular origin. The independent transgenic cells contributed to form the same TO shoots, so T1 plants from the same transgenic shoot could belong to different transgenic events.

TABLE 1
Selection and transformation frequency from cotton
ball-mediated infection of 3-weeks-old tobacco seedling
with Agrobacterium (OD A660 of 1)

| | | | | | T0 |
|---|---|---|---|---|---|
| Glyphosate | Infected | | | PCR | Transfor- |
| Selection | Seedling | Top | Bottom | positive | mation |
| strategy | number | selection | selection | events | frequency |
| Bottom | 20 | No | 300 uM: | 1 | 5% |
| selection | | | 6 weeks | | |
| 300 uM | | | | | |
| Bottom and | 20 | 50 uM: | 300 uM: | 2 | 10% |
| top selection | | three | 6 weeks | | |
| at the same | | weeks | | | |
| time | | | | | |
| Bottom | 20 | 50 uM: | 300 uM: | 4 | 25% |
| selection one | | 3 weeks | 5 weeks | | |
| week later | | | | | |
| after top | | | | | |
| selection | | | | | |

TABLE 2
Inheritance analysis of tobacco transgenic events in T1 generation.

| | Tested | T1 plant | T1 plant | | | |
|---|---|---|---|---|---|---|
| Event | T1 | containing | containing | T1 Plant(s): 1 | T1 Plant(2): 2 | T1 Plant(s): >2 |
| ID | plants | EPSPS gene | AmCyan gene | copy AmCyan | copies AmCyan | copies of AmCyan |
| 1 | 18 | 18 | 18 | 1 | 3 | 14 |
| 2 | 9 | 8 | 8 | 5 | 2 | 2 |
| 3 | 6 | 6 | 6 | 0 | 2 | 4 |
| 4 | 12 | 12 | 12 | 1 | 1 | 10 |
| 5 | 19 | 19 | 19 | 4 | 0 | 15 |
| 6 | 7 | 7 | 7 | 0 | 1 | 6 |
| 7 | 15 | 14 | 13 | 0 | 4 | 9 |

### Example 3: An Example Process for in Planta Transformation

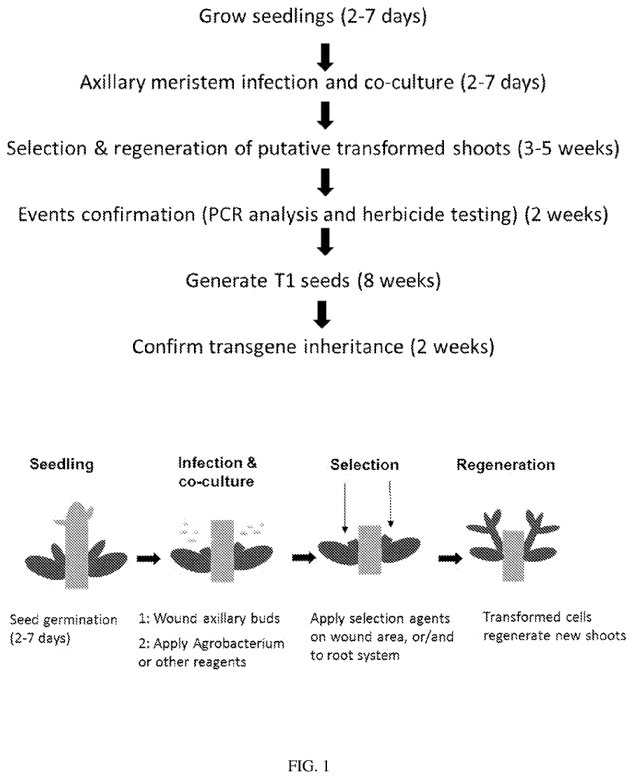

Another example general process for in planta transformation is outlined in FIG. 1 and a further example process is described below.

1) After seed germination for 1-7 days, one or both cotyledon meristems are removed or cotyledonary meristem area is wounded to create severe wounds. The plant apex is removed at the same time, or several days' later.

2) A transformation agent is used to facilitate transfer of the construct into the wounded meristem(s). An example is to apply Agrobacterium or virus containing construct onto the surface of the wounded area. Another example is to use biolistics or cell membrane penetrating peptides to introduce the construct.

3) After 1-7 days incubation, apply selection agents on the wounded areas (“top selection”), and/or water selection agent into soil/media (“bottom selection”). Keep plants under selection for 2-4 weeks, the transformed cells will develop into shoots.

5) Transplant potential transgenic plants into new soil pots and keep plants under good growth conditions. Verify positive transformants with molecular assays.

6) Grow the positives for (T1) seed production and identify germline transformed events from produced seeds.

### Example 4: In planta Transformation of Soy Seedlings with Construct Containing AmCyan and EPSPS

A construct containing AmCyan fluorescence and EPSPS selection genes was transformed into soybean using in planta transformation with Agrobacterium. The method of transformation is below.

1) Germination: Soybean seeds were sown in 2.5 inch pots, two seeds in each pot. 4-7 day old seedlings grown from the seeds were used for cotyledon axillary meristem transformation.

2) Wound axillary meristems: The axillary meristems located in the axil of cotyledons were completely removed. The stem cells located in the base of the axillary meristem were transformed using Agrobacterium as described below. To break the dormancy of the axillary meristem, the primary shoot meristem was removed at the same time, or 3-7 days' later.

3) Agrobacterium infection: Agrobacterium solution (OD=0.6-1.2) was applied on the wounded areas. The Agrobacterium contained the construct containing the AmCyan and EPSPS genes. For the infection, tiny cotton balls were soaked with Agrobacterium solution and mounted on the wounded areas for 0.5-24 hours (Table 3). For 7-day old seedlings, 5-12 hours of infection was found to result in better infection results.

TABLE 3
Infection times

| | | | The seedlings with | |
|---|---|---|---|---|
| | infection | | AmCyan signals | |
| Soybean | time | Infected | in 5 days after | Infection |
| variety | (hours) | seedlings | infection | rate |
| Elite Soybean | 0.5 | 45 | 9 | 20.0% |
| Variety A - | | | | |
| maturation | | | | |
| group 5 | | | | |
| Elite Soybean | 1 | 42 | 8 | 19.0% |
| Variety A | | | | |
| Elite Soybean | 3 | 48 | 16 | 33.3% |
| Variety A | | | | |
| Elite Soybean | 5 | 44 | 16 | 36.4% |
| Variety A | | | | |
| Elite Soybean | 7 | 47 | 18 | 38.3% |
| Variety A | | | | |
| Elite Soybean | 12 | 45 | 16 | 35.6% |
| Variety A | | | | |
| Elite Soybean | 24 | 48 | 15 | 31.3% |
| Variety A | | | | |

After infection, excess Agrobacterium solution was removed using a piece of filter paper or alternative absorptive materials.

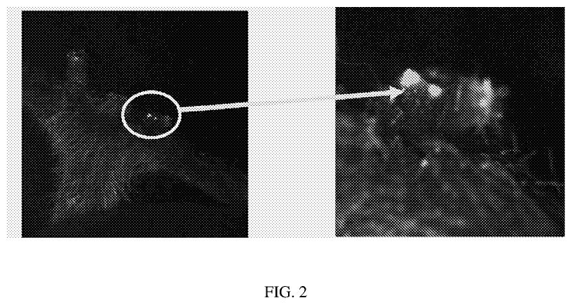

4) Incubation: Flat trays containing the infected seedlings were covered with a dome to keep in moisture. The seedlings were placed under darkness at a temperature of 22-25° C. AmCyan signals could be clearly observed at day-3 after infection, though the signals were weak. At day-5 and day-7, the signal intensity became much stronger. The seedlings were incubated with the Agrobacterium under darkness for 5 days . After incubation, antibiotics were applied (Timentin, Cefotaxime and Vancomycin) to inhibit the growth of Agrobacterium. FIG. 2 shows AmCyan signal in axillary meristem cells after incubation, confirming that this cell type can be transformed in planta.

5) Selection: To determine the glyphosate concentrations effectively suppressing axillary meristem regeneration in top selection, non-transgenic plants were used and 10 uM to 200 uM glyphosate (specifically 10, 25, 50, 75, 100, or 200 uM glyphosate) was applied on the wounded axillary areas. The results showed that 75-200 uM glyphosate were non-lethal but sufficient to suppress axillary meristem regeneration within 2 weeks.

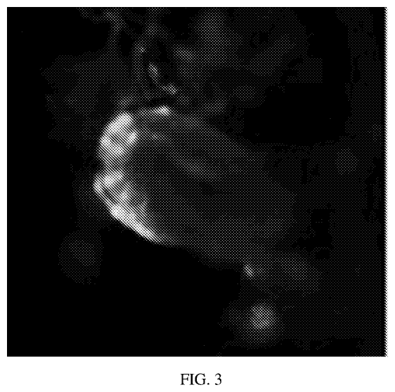

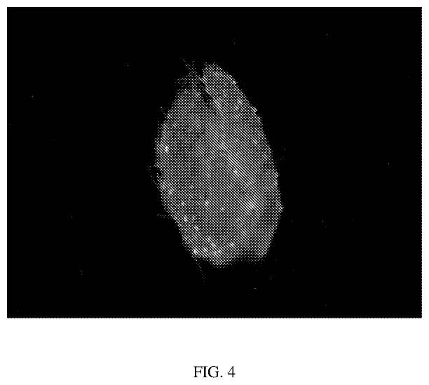

After incubation, the seedlings were moved into a growth chamber under 16 hours light: 8 hours dark conditions. A tiny cotton ball soaked in selection solution was mounted on the infected area (“top selection”). Top selection solution contained 75-150 uM glyphosate, 0.5-2 mg/L 6-Benzylaminopurine and 0.5-2 g/L 2-(N-morpholino) ethanesulfonic acid. The trays were covered with a dome to maintain high humidity. The cotton balls were changed 1-2 times weekly. Top selection lasted for 2 weeks. FIG.3 shows that AmCyan signal from the construct was detectable in transformed cells 7 days after top selection. FIG. 4 shows that AmCyan signal from the construct was detectable in a newly regenerated meristems 14 days after top selection.

Top selection was found to be very effective for soybean. However, the cotton balls can dry out, which may create some selection variation. To keep continuous selection pressure, glyphosate selection was watered into soil pots (“bottom selection”) for some plants. The selection watering containing 150-500 uM of glyphosate was applied once a week, for 4-5 weeks.

The putative transgenic shoots regenerated during 3-5 weeks after application of selection. The putative events were first identified based on their growth and leaf morphology. The putative transgenic shoots grew fast and had normal leaves. The non-transgenic shoots were stunted, grew slowly or had small and narrow leaves. Results of the transformation frequency are shown in Table 4. Bottom-selection only at 50, 100, 10, 200 and 300 uM glyphosate did not produce any positive events. Top-selection-only at 75 uM glyphosate did not produce any positive events.

TABLE 4
Summary of transformation frequency with
different selection conditions tested.

| | | Glyphosate | Glyphosate | CFP | |
|---|---|---|---|---|---|
| | Infected | for top | for bottom | positive | Transfor- |
| | seedling | selection | selection | event | mation |
| Germplasm | No. | (uM) | (uM) | No. | Frequency |
| Elite | 80 | 100 | 0 | 1 | 1% |
| Soybean | | | | | |
| Variety A | | | | | |
| Elite | 31 | 125 | 0 | 2 | 1.6% |
| Soybean | | | | | |
| Variety A | | | | | |
| Elite | 45 | 100 | 500 | 1 | 2% |
| Soybean | | | | | |
| Variety A | | | | | |
| Elite | 30 | 100 | 300 | 2 | 6% |
| Soybean | | | | | |
| Variety A | | | | | |

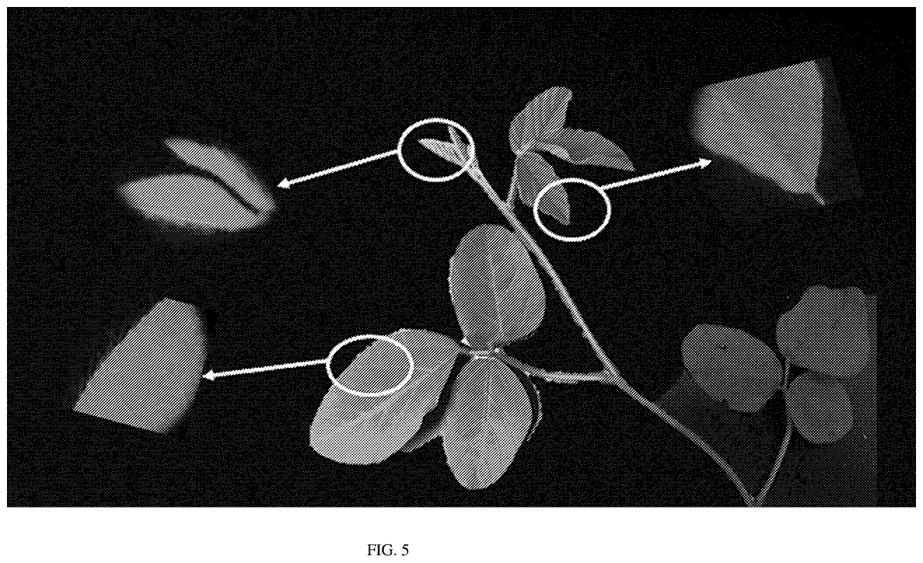

6): Transformation confirmation of putative transgenic shoots: Two approaches were used to identify putative transgenic shoots. One approach was to observe AmCyan signals under fluorescence microscope. As shown in FIG. 5, AmCyan signal was distributed evenly across different leaves thought to be putatively transgenic. Another approach was to use real time PCR to identify the transgenes in the plant tissue. Both AmCyan and EPSPS transgenes were detectable by real time PCR across the tissues derived from this transgenic shoot. These data show successful in planta transformation at the TO stage. 7): Test in planta transformation approach in different germplasms.

Because this approach avoids the traditional regeneration process it is expected that this method will achieve a genotype-independent protocol for plant transformation. Because the process is fast, it can be used for transformation of some early maturation soybean germplasms. To test this hypothesis, side-by-side experiments were conducted with three soybean germplasms representing different maturity groups This MG2 elite line was very difficult to generate healthy TO seedlings using tissue-culture transgenic approach because of early flowering and senescence in medium plates. To test this hypothesis, the three lines were transformed using this method. Both top and bottom selection lasted for 4 weeks. The results, shown in Table, 5 indicated the transformation events can be generated across three different germplasms.

TABLE 5
In planta transformation in different germplasms

| | | Glyphosate | Glyphosate | CFP | |
|---|---|---|---|---|---|
| | Infected | for top | for bottom | positive | Transfor- |
| | seedling | selection | selection | event | mation |
| Germplasm | No. | (uM) | (uM) | No. | Frequency |
| Jack | 41 | 100 | 300 | 6 | 14.6% |
| Maturity | | | | | |
| group 2 | | | | | |
| Elite | 40 | 100 | 300 | 2 | 5% |
| Soybean | | | | | |
| Variety A - | | | | | |
| maturation | | | | | |
| group 5 | | | | | |
| Elite | 43 | 100 | 300 | 3 | 6.9% |
| Soybean | | | | | |
| Variety B - | | | | | |
| maturation | | | | | |
| group 2 | | | | | |

8): Germline transformation confirmation:

After transformation, transgenic shoots were grown to maturity and T1 seeds were generated. Progeny analysis of TO transformants was carried out by PCR amplification of EPSPS and AmCyan genes. The four earliest events in T1 generation were tested and the transgenes were detected in T1 seeds in two events.

### Example 5: Inheritance of Transgene in Soybean

The transgene AmCyan is present in construct 23093. All transgenic plants generated from this construct are expected to carry both visible marker gene, AmCyan, and selectable marker gene, EPSPS. The inheritance of transgene can be demonstrated by PCR analysis of AmCyan and EPSPS genes. We selected 14 to15 events from each germplasm to determine the inheritance of the transgene. Ten T1 plants per event were analyzed via PCR. Table 6 summarized the analysis results. The results demonstrated the inheritable transmission of the transgenes from TO to T1 generation. The transgenes were not detected in some events, indicating the presence of chimera. The chimeric transformation can be reduced via selection optimization.

TABLE 6
Transgene inheritance in T1 progeny

| | | Average | | | |
|---|---|---|---|---|---|
| | | number of | | | Inheri- |
| | Number | T1 plants/ | CFP | EPSPS | tance |
| | of Events | event | positive | positive | rate in T1 |
| Germplasm | analyzed | analyzed | events | events | generation |
| Jack | 15 | 10 | 8 | 8 | 53% |
| Elite | 14 | 10 | 11 | 11 | 78% |
| Soybean | | | | | |
| Variety A - | | | | | |
| maturation | | | | | |
| group 5 | | | | | |
| Elite | 14 | 10 | 9 | 9 | 64% |
| Soybean | | | | | |
| Variety B - | | | | | |
| maturation | | | | | |
| group 2 | | | | | |

To evaluate the inheritance of transgenes in T2 generation, we selected one T1 homozygous plant per event and generated T2 seeds. PCR analysis confirmed stable transgenes in T2.

TABLE 7
Transgene inheritance in T2 soybean progeny

| | Number of | Homozygous T1 | Analyzed T2 | EPSPS | ALS gene | Inheritance |
|---|---|---|---|---|---|---|
| | heritable T0 | plants selected | plants | detected in | detected in | rate from |
| Germplasm | Events selected | per event | per event | all T2 plants | all T2 plants | T1 to T2 |
| Jack | 8 | 1 | 10 | yes | yes | 100% |
| Elite Soybean Variety A - | 11 | 1 | 10 | yes | yes | 100% |
| maturation group 5 | | | | | | |
| Elite Soybean Variety B - | 6 | 1 | 10 | yes | yes | 100% |
| maturation group 2 | | | | | | |

### Example 6: In Planta Transformation of Soybean with a Construct Containing AmCyan and ALS genes using Top-Selection only Method

Method

1. Soybean Seedling Preparation:

Soybean (Glycine max) seeds were pre-germinated on paper towels soaked with 2 mg/L BAP solution for 24 hours at room temperature. The pre-germinated seeds were sown in 2.5-inch pots with two seeds in each pot. Pots were placed in a flat and each flat held 32 pots. 3-6 day old seedlings were used for cotyledon axillary meristem transformation.

2. Agrobacterium Suspension Preparation: Agrobacterium tumefaciens strain [Chry5d3 recA-] was used. The Agrobacterium was transformed with a binary vector containing a selectable marker gene Acetolactate synthase (ALS) and an AmCyan fluorescence protein (CFP) gene. Agrobacterium cells were suspended in liquid infection medium containing 1.1 g/L MS basal salt mixture, 20 g/L sucrose, 10 g/L glucose, 4 g/L MES, 1 ml/L Gamborg's B5 vitamins (1000×) and 2 mg/L zeatin riboside. A final concentration of 40-80 mg/L (200-400 uM) acetosyringone was added to induce virulence gene expression. Dithiothreitol (DTT) was added to a final concentration of 150 μg/ml.

3. Agrobacterium-mediated infection and co-culture: The axillary buds located in the axil of cotyledons were completely removed by a blade. To break the apex dominance, the primary shoot meristem was removed at the same time. For the infection, tiny cotton balls soaked with Agrobacterium solution (OD=0.5-1) were mounted on the wounded area for 5-24 hours. After infection, excess Agrobacterium solution was removed using a piece of filter paper or alternative absorptive material. Flat trays containing the infected seedlings were covered with a dome. Seedlings were placed under darkness at a temperature of 22 ° C. for 3-5 days. After co-culture, antibiotics were applied (Timentin, Cefotaxime and Vancomycin) to inhibit the growth of Agrobacterium.

4. Selection:

1) Week-1 selection: After co-culture, seedlings were moved into a growth chamber under a16-hour light, 8-hour dark condition. A tiny cotton ball was soaked in selection solution and mounted on the infected area (“top selection”). Top selection solution contains 2 mg/L BAP, 1 g/L 2-(N-morpholino) ethanesulfonic acid (MES), 2-7 uM bensulfuron-methyl, 1 uM 3.1 g/L Gamborg's B5 basal medium, 5 ml MS iron (200×), 1 ml/L Gamborg's B5 vitamins (1000×), 100 mg/L glutamine, 100 mg/L asparagine, 300 mg/L timentin. The flat was covered with a dome to keep the moisture. Fresh selection solution was applied daily to keep the cotton wet. After 7 days, cotton balls were removed.

2) Week 2-4 selection: The selection was performed by spraying selection solution, which contains 3-7 uM bensulfuron-methyl, 1 mg/L BAP (6-Benzylaminopurine), and 1 g/L MES (2-(N-morpholino) ethanesulfonic acid. Seedlings in each tray were sprayed once a day with 50 ml of selection solution for 2-3 weeks. Regenerated shoots were then sampled for Taqman assay.

### Results

Results are shown in Table 9 and Table 10. These results demonstrated that in planta soybean transformation method can also work using different selectable marker other than EPSPS. The heritable transformation was achieved across multiple soybean germplasm lines through top selection process.

TABLE 8
In planta transformation of soybean with a CFP + ALS construct and top selection

| | | | Top | Top | PCR | |
|---|---|---|---|---|---|---|
| Experiment | | Infected | Selection | selection | positive | Transformation |
| ID | Germplasm | Seedlings | week 1 | Week 2-3 | T0 events | Frequency |
| A-1 | Elite Soybean Variety A - | 51 | 2 uM | 3 uM | 0 | 0 |
| | maturation group 5 | | | | | |
| A-2 | Elite Soybean Variety A - | 49 | 3 uM | 3 uM | 0 | 0 |
| | maturation group 5 | | | | | |
| A-3 | Elite Soybean Variety A - | 109 | 3 uM | 7 uM | 4 | 3.7% |
| | maturation group 5 | | | | | |
| A-4 | Elite Soybean Variety A - | 112 | 4 uM | 7 uM | 6 | 5.4% |
| | maturation group 5 | | | | | |
| A-5 | Elite Soybean Variety A - | 103 | 4 uM | 7 uM | 5 | 4.9% |
| | maturation group 5 | | | | | |
| A-6 | Elite Soybean Variety A - | 50 | 4 uM | 7 uM | 2 | 4% |
| | maturation group 5 | | | | | |
| J-1 | Jack | 50 | 4 uM | 7 uM | 1 | 2% |
| J-2 | Jack | 46 | 4 uM | 7 uM | 1 | 2.2% |
| OW-1 | Elite Soybean Variety B - | 56 | 4 uM | 7 uM | 1 | 1.8% |
| | maturation group 2 | | | | | |
| OW-2 | Elite Soybean Variety B - | 50 | 4 uM | 7 uM | 1 | 2% |
| | maturation group 2 | | | | | |

TABLE 9
Inheritance analysis of transgenes in soybean
events recovered from in planta transformation
using ALS selection and top selection method

| | | Average | | | |
|---|---|---|---|---|---|
| | | number of | | | Inheri- |
| | Number | T1 plants/ | CFP | EPSPS | tance |
| | of Events | event | positive | positive | rate in T1 |
| Germplasm | analyzed | analyzed | events | events | generation |
| Jack | 2 | 10 | 2 | 2 | 100% |
| Elite | 11 | 10 | 7 | 9 | 63%-82% |
| Soybean | | | | | |
| Variety A - | | | | | |
| maturation | | | | | |
| group 5 | | | | | |
| Elite | 2 | 10 | 2 | 2 | 100% |
| Soybean | | | | | |
| Variety B - | | | | | |
| maturation | | | | | |
| group 2 | | | | | |

### Example 8: In Planta Transformation of Tobacco (Nicotiana benthamiana) Seedlings in Planta with a Different Selection Maker other than EPSPS

### Methods

1. Preparation of tobacco seedlings for transformation: Tobacco seeds were sown in 2.5 inch soil pots. After germination, any additional plants were removed so that there was only one seedling per pot. Three-week old seedlings were used for axillary meristem transformation.
2. Agrobacterium strain [Chry5d3 recA-] was transformed with a binary vector containing a selectable marker gene Acetolactate synthase (ALS) and an AmCyan fluorescence protein (CFP) gene. Agrobacterium cells were cultured in liquid infection medium containing 1.1 g/L MS basal salt mixture, 20g/L sucrose, 10 g/L glucose, 4 g/L MES, 1 ml/L Gamborg's B5 vitamins (1000×) and 2 mg/L zeatin riboside. A final concentration of 40-80 mg/L (200-400 uM) acetosyringone was added to induce virulence gene expression. Dithiothreitol (DTT) was added to a final concentration of 150 μg/ml.
3. Removal of axillary meristems and stem apex: Axillary buds in each leaf axil was removed using a blade. The shoot apex meristem was then completely removed to eliminate apex dominancy
4. Infection and Co-culture: For the infection, tiny cotton balls were soaked with Agrobacterium solution and mounted on the wounded areas for 7 hours. After infection, excess Agrobacterium solution was removed using a piece of filter paper or paper towel. After infection, flat trays containing the infected seedlings were covered with a dome to keep the moisture. Seedlings were placed in a growth chamber at 22-25 ° C-under darkness for 3-7 days.
5. Selection: After co-culture, antibiotics were applied (Timentin, Cefotaxime and Vancomycin) to inhibit the growth of Agrobacterium. Seedlings were moved into a growth chamber under a16-hour light and 8-hour dark condition. Then a tiny cotton ball was soaked in selection solution containing 0.5 uM bensulfuron-methyl, 1 g/L MES, and 0.5-1 mg/L BAP (6-Benzylaminopurine) and mounted on the infected axil area. Trays were covered with a dome to maintain high humidity. The cotton balls were changed 1-2 times weekly. After two weeks, bensulfuron-methyl concentration was increased to lum. The selection was lasted for 4-7 weeks and then terminated after the adventitious shoots were generated.
6. Molecular analysis to identify the transgenic shoots: Leaf tissues were sampled from putative transgenic shoots, and then DNA was extracted and analyzed for the presence of transgenes. Positive events were transplanted into new soil pots and grown under a16-hour day-length condition until T1 seeds were harvested.
7. T1 seeds were sown and the transgene presence was detected in T1 generation based on CFP signals and molecular approach.

Results

The results of the above method are shown in Table 10. Tobacco seedlings were used for transformation. One shoot was generated from the process. PCR confirmed the presence of AmCyan and ALS genes and the transgenes are single copy. To validate the heritable transformation, we observed 51 T1 seedlings under a fluorescence microscope and found 37 plants showed CFP signals.

TABLE 10
Transgene inheritance in tobacco T1 and T2 progenies

| | PCR | | | Number | Number of |
|---|---|---|---|---|---|
| Infected | positive | Transfor- | Event | of T1 | T1 plants |
| Seedling | events | mation | copy | plants for | containing |
| number | (T0) | efficiency | number | analysis | CFP gene |
| 10 | 1 | 10% | 1 | 51 | 37 |

### Example 9: In Planta Transformation of Sunflowers (Helianthus annuus)

### Method

1. Sunflower seedling preparation:
Sunflower seeds (germplasm F75400) were sown in 2.5 inch pots with one seed per pot, and each flat held 32 pots. 5-7day old seedlings were used for in planta transformation.
2. Agrobacterium-mediated infection and co-culture: Agrobacterium was transformed with a binary vector containing a selectable marker gene Acetolactate synthase (ALS) and an AmCyan fluorescence protein (CFP) gene. The cotyledonary axillary buds and the stem apex were completely removed by a blade. For the infection, tiny cotton balls were soaked with Agrobacterium solution (0D=0.5-1) and mounted on the wounded areas for 5-24 hours. After infection, cotton balls were removed and seedlings were grown under darkness at 25 ° C. for 3-5 days.
3: Selection: After co-culture, seedlings were moved into a growth chamber under 16- hour light and 8-hour dark conditions. A tiny cotton ball was soaked in selection solution and mounted on the infected area (“top selection”). Top selection solution contained 0.5-3 uM bensulfuron-methyl, 1-2 mg/L BAP (6-Benzylaminopurine), and 1 g/L MES (2-(N-morpholino) ethanesulfonic acid.

### Results

We have observed the CFP signals after Agrobacterium infection and culture, which indicated the infection process works. We had success in organogenesis of sunflower adventitious shoots from cotyoledonary areas, and the regenerated shoots produced normal heads and seeds. Our results indicated the regeneration system works well using this method. We have not got transgenic plants yet, but we expect to make a success after development of an effective selection protocol for sunflower.

### Example 10: An Example Process for in Planta Transformation of Recalcitrant Plants

Dicot embryos include an epicotyl (shoot apical meristem), a radicle, a hypocotyl and two cotyledons. However, this is another very tiny structure located at the axil between each cotyledon and the shoot. These structures are axillary meristems and are referred to as cotyledon axillary meristems (or cotyledon axillary buds at seedling stage). Such cotyledon buds have strong meristematic abilities, especially when the main shoot is removed, thus providing excellent materials for in planta transformation, which can be used for genome editing and transgenic plant generation.
A first example process for in planta is described below.
1) After seed germination or plant in seedling stage, remove or damage axillary meristems located in the axil of cotyledons or true leaves to create severe wounds. To break the dormancy of the axillary meristem, the primary shoot meristem is removed at the same time, or several days' later.
2) A transformation agent is used to facilitate transfer of the construct into the wounded meristem(s). An example is to apply Agrobacterium. A first Agrobacterium strain is transformed with a binary vector containing an expression cassette driving the expression of a gene of interest or genome editing machinery. A second Agrobacterium strain is also included. This second Agrobacterium is transformed with a binary vector containing an expression cassette driving the expression of a morphogenic factor (MF) or a developmental regulator (DR) such as Baby Boom (BBM), Wuschel (WUS/Wox), Growth-Regulating Factor (GRF), Growth-Regulating Factor 4 GRF4) and its cofactor GRF-Interacting Factor 1 (GIF1), Shoot. Meristemless (STM) or Isopentenyl Transferase (IPT). Expression of the MF/DR improves transformation of recalcitrant plants through de novo meristem induction. A second expression cassette drives 1) pollen specific expression of barnase selecting against gametes with the co-transformed MF/DR transgene thereof, or 2) fluorescent marker genes expressed in seeds, embryos or seedlings allowing the identification and removal of events with these MF/DR transgene in the gene of interest (GOI)/genome edited (GE) progeny.
3) After 1-7 days incubation under darkness or weak light, some cells within wounded meristem tissues will be transformed. Apply negative or positive selection agents to suppress non-transformed cell division and facilitate transformed cell proliferation. The selection agents are applied on the wounded areas (“top selection”), and/or watered into plant growth media or soil (“bottom selection”).
4) Keep plants under selection conditions for several weeks. The transformed cells proliferate and develop into transgenic shoots, while the non-transgenic shoots are suppressed by selection and severely stunted. The transformants can be verified with molecular- or bio-assays.
5) Grow the positives for (T1) seed production and identify germline transformation from produced seeds.

In summary, the results in these Examples show that in planta transformation methods can be used in multiple plant types to produce transgenic shoots, which can then generate T1 transgenic seeds. These transformation methods were also shown to be effective for transforming different germplasms and elite germplasm such that these methods are expected to be genotype-independent and useful for germplasm, such as elite germplasm, that otherwise is difficult to transform by more conventional transformation methods.

### REFERENCES

U.S. Pat. No. 5,376,543

U.S. Pat. No. 5,015,580

U.S. Pat. No. 7,002,058

US20020073445

US2003046733

US2004034889

US2005268357

US20040237133

WO0042207

WO05121345

CN103667342A

CN106399359A

Li et al. Optimization of Agrobacterium-Mediated Genetic Transformation System of

Soybean Cotyledonary Node with Non Tissue-Culture. 2013. Journal of Plant Genetic Resources, Vol. 13, No. 5, pp. 789-797.

Janani et al. Construction and transformation of peroxisome proliferator activated receptor gamma (RnPPARy) gene using Agrobacterium tumefaciens into Glycine max L. Merr. 2019. Gene Reports, Vol. 16, p. 100427.

Mangena et al. Challenges of In Vitro and In Vivo Agrobacterium-Mediated Genetic Transformation in Soybean, Soybean—The Basis of Yield, Biomass and Productivity, Minobu Kasai, IntechOpen, DOI: 10.5772/66708. 2017. Available from: www.intechopen.com/books/soybean-the-basis-of-yield-biomass-and-productivity/challenges-of-in-vitro-and-in-vivo-agrobacterium-mediated-genetic-transformation-in-soybean

Soto et al. Efficient particle bombardment-mediated transformation of Cuban soybean (INCASoy-36) using glyphosate as a selective agent. 2017. Plant Cell, Tissue and Organ Culture, Vol. 128, No. 1, pp. 187-196. strike-through and adding the underlined material. This listing of claims will replace all prior versions and listings of the claims in this application.

1. A method, comprising:
a) Providing a plant comprising an axillary meristem and a shoot apical meristem,
b) Wounding at least part of the axillary meristem to produce a wounded axillary meristem region,
c) Contacting the wounded axillary meristem region with a heterologous polynucleotide and/or heterologous protein under conditions where the heterologous polynucleotide and/or heterologous protein enters wounded axillary meristem region,
d) Removing the shoot apical meristem or suppressing the growth of the shoot apical meristem at the same time as step b) or step c) or after step c), and
e) Growing the plant to regenerate at least part of the wounded axillary meristem region to produce a regenerated axillary meristem or shoot.

2. The method of claim 1, wherein the axillary meristem is two axillary meristems, the wounded axillary meristem area is two wounded axillary meristem areas, and the regenerated axillary meristem is two regenerated axillary meristems.

3. The method of claim 1, wherein the method comprises removing or suppressing the shoot apical meristem at the same time as step b).

4. The method of claim 1, wherein the method comprises removing or suppressing the shoot apical meristem after step c).

5. The method of claim 4, wherein the shoot apical meristem is removed or suppressed 2-7 days, optionally 3-4 days, after the contacting.

6. The method of claim 1, wherein the plant is a dicot plant, optionally a soy plant, a tobacco plant, a bean plant, a sunflower plant, a cotton plant, a tomato plant, a watermelon plant, a squash plant, a cucumber plant, a lettuce plant or a pepper plant.

7. The method of claim 1, wherein step c) comprises contacting the wounded axillary meristem region with a heterologous polynucleotide, wherein the heterologous polynucleotide comprises a selectable marker and wherein the method further comprises contacting the plant with a selection agent to eliminate or reduce untransformed tissue, wherein at least part of the contacting with the selection agent occurs during or after step e).

8. The method of claim 7, wherein the contacting with the selection agent comprises (i) adding the selection agent to a medium in which the plant is growing, (ii) spraying the plant with the selection agent, or (iii) applying the selection agent to the wounded axillary meristem region and/or regenerated axillary meristem, or a combination thereof, optionally wherein the combination thereof is a combination of (i) and (iii).

9. The method of claim 7, wherein the contacting with the selection agent occurs for a period of time, optionally for at least one week, further optionally between 3-5 weeks.

10. The method of claim 7, wherein the selection agent is an herbicide, an antibiotic, or a non-metabolizable sugar.

11. The method of claim 7, wherein the selection agent is glyphosate, glufosinate, spectinomycin, bensulfuron-methyl, imazapyr, D-xylose, mannose or kanamycin.

12. The method of claim 1, wherein the method further comprises performing an assay on the regenerated axillary meristem or a sample of the regenerated axillary meristem to assess for the presence or absence of transformed cells and/or to assess for the number of transformed cells.

13. The method of claim 1, wherein the method further comprises growing the plant to produce a seed and harvesting the seed, wherein the seed optionally comprises at least part of the heterologous polynucleotide.

14. The method of claim 13, wherein the method further comprises growing the seed to produce a progeny plant, optionally wherein the progeny plant comprises at least part of the heterologous polynucleotide.

15. The method of claim 1, wherein the heterologous polynucleotide encodes or comprises a genome editing agent or wherein the heterologous protein comprises a genome editing agent, optionally wherein the genome editing agent is a nuclease or a recombinase.

16. The method of claim 15, wherein the heterologous polynucleotide comprises one or more polynucleotides encoding a Cas protein and/or a guide RNA or wherein the heterologous protein comprises a Cas protein, optionally wherein the Cas protein is Cas9 or Cas12a, or a functional variant thereof.

17. The method of claim 1, wherein the heterologous polynucleotide comprises an expression cassette comprising a coding sequence.

18. The method of claim 17, wherein the expression cassette further comprises a promoter operably linked to the coding sequence.

19. The method of claim 17, wherein the coding sequence encodes a protein or non-coding RNA of interest.

20. The method of claim 1, wherein the contacting in step c) is performed with Agrobacterium, viral particles, microparticles, nanoparticles, cell membrane penetrating peptides, aerosol beam, chemicals, electroporation, or pressure.

21. The method of claim 20, wherein the contacting is performed with Agrobacterium or viral particles and the contacting comprises an infection step and an incubation step.

22. The method of claim 21, wherein the infection step is performed for 30 minutes to 24 hours, optionally 1-9 or 5-12 hours, and the incubation step is performed in darkness for at least 2 days, optionally 3-7 days.

23. The method of claim 1, wherein the plant is between 1-30 days old, optionally 4-7 days old.

24. The method of claim 1, wherein the axillary meristem is a cotyledonary axillary bud, or a meristem in an axil of a true leaf.

25. The method of claim 1, , wherein the method further comprises removing a cotyledon of the plant prior to removing or suppressing the shoot apical meristem.

26. The method of claim 1, wherein the method further comprises growing the regenerated axillary meristem into a shoot.

27. A plant or plant part produced by the method of claim 1.

28. A progeny seed produced by crossing the plant of claim 27 with a second plant or by selfing the plant of claim 27.

29. A derivative or a commodity product produced or obtained from the plant or plant part of claim 27.
